(19) United States
(12) Patent Application Publication
(10) Pub. No.: US 2017/0000678 A1
(43) Pub. Date: Jan. 5, 2017

(54) Ergodont Dental System

(71) Applicants: KATIE JACOBSON, NAMPA, ID (US); ROLINA SCHNUERLE, BOISE, ID (US); JEFF THALDORF, BOISE, ID (US)

(72) Inventors: KATIE JACOBSON, NAMPA, ID (US); ROLINA SCHNUERLE, BOISE, ID (US); JEFF THALDORF, BOISE, ID (US)

(21) Appl. No.: 15/200,673

(22) Filed: Jul. 1, 2016

Related U.S. Application Data
(60) Provisional application No. 62/187,537, filed on Jul. 1, 2015.

(51) Int. Cl. A61G 15/16; A61B 1/05; A61B 1/00; A61G 15/02; A61B 1/24

(52) U.S. Cl. CPC A61G 15/16; A61G 15/02; A61B 1/24; A61B 1/00045; A61B 1/00009; A61B 1/05; A61B 2560/0425

(57) ABSTRACT

A system for enabling a clinician to perform treatments on a patient while the clinician maintains an ergonomically sound position. The system includes a case that is mountable to a chair, an articulating arm that supports a display, a flexible camera that captures images of a region of interest and transmits them to the display. Disclosed embodiments may also include flexible tool mounts that holster tools that can be used during the treatment.

### CROSS-REFERENCE TO RELATED APPLICATIONS

This application, under 35 U.S.C. §119, claims the benefit of U.S. Provisional Patent Application Ser. No. 62/187,537 filed on Jul. 01, 2015, and entitled “Ergospective Camera,” the contents of which are hereby incorporated by reference herein.

### FIELD OF THE DISCLOSURE

This disclosure relates to ergonomic tools to facilitate treatment of a patient. In particular, the disclosure relates to a kit of dental tools that facilitate proper ergonomic positioning of the clinician while performing dental procedures.

### BACKGROUND

As used throughout this disclosure a “clinician” may be a doctor, nurse, dentist, dental hygienist, dental assistant, orthodontist, orthodontist's assistant, periodontist, pedodontist, prosthodontist, oral surgeon, endodontist, cosmetologist, make-up artist, dermatologist, esthetician, or the like, that perform treatments or procedures on patients, clients, customers, or the like. As used throughout this disclosure a “treatment” or “procedure” is any of the services, treatments, procedures, or the like that a clinician may perform. As also used throughout this disclosure a “patient” is the person upon whom the clinician performs a treatment whether or not the treatment is medical in nature.

Traditional dental procedures typically require a patient to be seated in a reclining chair, or the like, to position the patient's mouth in an accessible position for treatment. However, use of such a chair requires the clinician to be alongside, or behind, the patient and, consequently, causes the clinician to bend and twist repeatedly, and for prolonged durations, in order to perform the treatment. This prolonged, repeated twisting and bending may lead to may musculoskeletal issues, such as lower back pain, neck pain, damaged vertebrae, spine issues, vertebral disk issues, carpal tunnel issues, cramped muscles of the neck, back, and/or shoulders, or the like. In fact, some studies document that approximately 87% of clinicians have reported at least one symptom of musculoskeletal issue.

In addition, many clinicians perform their services at more than one location, and each location may have diverse equipment that makes it difficult or inconvenient for the clinician to work in a comfortable, efficient manner. Accordingly, there is a need for a portable kit of tools that the clinician can carry from location to location in order to improve the comfort and efficiency of the clinician's work.

Further, while many clinicians wear a loupe or other optical aid to assist the clinician in viewing the area of interest, loupes are often heavy (which can exacerbate the musculoskeletal issues), inconvenient to adjust, and may not significantly address the need for the clinician to bend and twist during treatment. Therefore, there is a need for a more convenient and ergonomic solution.

Additionally, some existing systems use a wall or ceiling mounted microscope to assist with “microscopic dentistry” procedures. However, such devices are typically a large piece of equipment that covers the patient's face, which makes it difficult for the clinician to make eye contact with the patient during the procedure. Likewise, existing systems provide a limited view of oral cavity.

In addition, existing perioscopes, or perioscopy procedures provide illumination into the periodontal pocket, but do not address the musculoskeletal problems associated with practicing clinical dental hygiene since the camera is attached to a hand instrument and the clinician is still twisting and looking down in an awkward position for extended periods of time (e.g., 8 or more hours a day). Further, existing intra-oral cameras are bulky, obtuse and difficult to use by one clinician, an assistant is needed to try to capture the image in a clear fashion. Because they are handheld, there is typically some amount of hand “shake” and images are often blurry. Also, existing systems create image that are usually upside down or slightly blurred. Other drawbacks with existing systems and methods also exist.

### SUMMARY

Accordingly, the above and other drawbacks and issues with existing systems are addressed by the disclosed embodiments. Disclosed embodiments include a system having a case that is mountable to a chair, an articulating arm that, when not in use, fits within the case and, when in use, may be attached to the chair and supports a display, a positionable camera that, when not in use, fits within the case and, when in use, may be positioned adjacent the chair to capture images of a region of interest, and wherein the positionable camera transmits images of the region of interest to the display.

Further disclosed embodiments include a mounting plate that attaches to the case and enables the case to be mounted on the chair. Still further embodiments include a clamp that attaches the mounting plate to the chair.

Disclosed embodiments also include a flexible tool mount that, when not in use, fits within the case and, when in use, is positionable adjacent the chair and holsters a tool.

In some embodiments, the display comprises a general purpose tablet computer. In further embodiments, the display further comprises programming for image processing.

In some embodiments, the positionable camera comprises an endoscopic camera. In further disclosed embodiments, the endoscopic camera further comprises a distal camera and a medial camera. In further embodiments, the distal camera and the medial camera are rotatable about a rotatable joint.

Disclosed embodiments also include a system for ergonomic performance of dental procedures comprising a case that is mountable to a dentist chair, an articulating arm that, when not in use, fits within the case and, when in use, may be attached to the dentist chair and supports a display in an ergonomically sound location, a positionable camera that, when not in use, fits within the case and, when in use, may be positioned adjacent the dentist chair to capture images of a patient's mouth, and wherein the positionable camera transmits images of the patient's mouth to the display.

Further embodiments include a mounting plate that attaches to the case and enables the case to be mounted on the dentist chair. In still further embodiments, a clamp attaches the mounting plate to the dentist chair.

Some embodiments may include a flexible tool mount that, when not in use, fits within the case and, when in use, is positionable adjacent the dentist chair and holsters a dental tool.

Disclosed embodiments include a display that comprises a general purpose tablet computer. In some embodiments, the display further comprises programming for image processing.

In some embodiments, the positionable camera comprises an endoscopic camera. In further embodiments, the endoscopic camera further comprises a distal camera and a medial camera. In further embodiments, the distal camera and the medial camera are rotatable about a rotatable joint. Other embodiments also exist.

### BRIEF DESCRIPTION OF THE DRAWINGS

FIG. 1 is a schematic side view of the system in accordance with disclosed embodiments.

FIG. 2 is a schematic back view of a portion of the system in accordance with disclosed embodiments.

FIG. 3 is a schematic side view of the system in accordance with disclosed embodiments.

FIG. 4 is a schematic top-down, inside view of a case for the system in accordance with disclosed embodiments.

FIG. 5 is a schematic representation of a camera in accordance with disclosed embodiments.

FIG. 6 is a schematic representation of the system integrated into a chair in accordance with disclosed embodiments.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

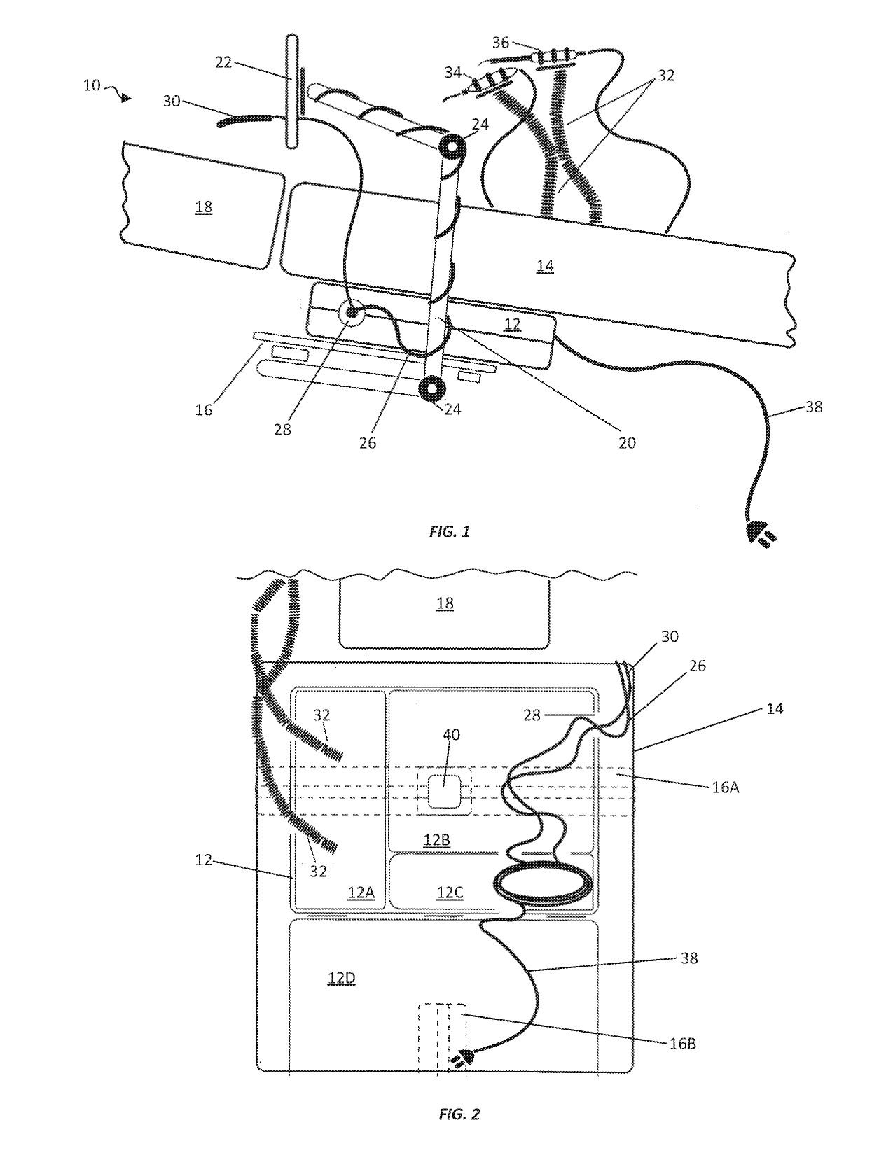

FIG. 1 is a schematic side view of the system in accordance with disclosed embodiments. As shown, the Ergodont system 10 may include a case 12 mounted to a chair 14 via mounting plate 16. As shown in this embodiment case 12 may be mounted to chair 14 in a closed position. Case 12 may be formed of any suitable material. For example, case 12 may be molded plastic, metal, wood, leather, composite material, or combinations of the same. Case 12 houses the other items of the Ergodont kit as disclosed herein and can be used to transport the system 10 from location to location. Therefore, case 12 is made of suitably durable and lightweight materials to allow a clinician to easily and safely transport the system 10.

Chair 14 may comprise any suitable clinician's chair. For example, chair 14 may be a dentist's chair, a reclining chair, a barber's chair, a beautician's chair, a doctor's examination table, a massage table, or the like. In some embodiments, chair 14 is not part of the portable system 10, but is a pre-existing chair installed in the place where the clinician is performing a treatment. In other embodiments, the system 10 may be installed in, or built-in, the chair 14 in a more permanent fashion. As also shown in FIG. 1, some embodiments of chair 14 may also include a headrest 18 to support the patient's head during a procedure.

Mounting plate 16 may comprise any suitable plate, bracket, or support that, among other things, enables the case 12, and other system components as described below, to be securely and conveniently mounted to chair 14. As discussed below, mounting plate may comprise one or more component pieces and may attach to chair 14 in any suitable fashion to enable the secure support of case 12 and the other system 10 components. For example, screws, bolts, or other selectively releasable fasteners may be used to attach mounting pate 16 to chair 14. In some embodiments, clamps 42, straps, hook-and-loop strips, or the like may be used alone, or in conjunction with mounting plate 16 to secure the case 12 to the chair 14. In embodiments, where system 10 is integral with the chair 14, mounting plate 16 may not be necessary, or may be an internal component of chair 14.

Mounting plate 16 may comprise any suitable material sufficient to reliably and durably function to secure case 12 to chair 14. For example, mounting plate 16 may comprise metals, composites, Plexiglas, fiberglass, or other polymer-based material, plastics, wood, or combinations of the foregoing.

As also shown in FIG. 1, system 10 may include an articulating arm 20 that attaches to case 12, or mounting plate 16 (as shown in FIG. 1), and can be used by the clinician to position a display 22 at an ergonomically favorable location. Articulating arm 20 may comprise one or more joints 24 that are selectively positionable to enable the clinician to position the display 22 at an ergonomically favorable location (e.g., so that the clinician does not need to bend or twist to view the display 22). In some embodiments, joints 24 may be omitted and articulating arm 20 may comprise flexible, jointed tubing, or the like, (e.g., gooseneck tubing) that also allows positioning and support of the display 22. Articulating arm 20 also includes a bracket, clip, or the like to support display 22.

As also shown in FIG. 1, system 10 may include a cable 26 that emanates from a port 28 in case 12. Cable 26 may provide power and communication of data to display 22. As shown in FIG. 1, cable 26 may spiral around articulating arm 20, or for embodiments where articulating arm 20 is hollow, cable 26 may be contained with articulating arm 20.

Display 22 may comprise any suitable display device capable of displaying images from camera 30. For example, display 22 may comprise a general purpose tablet computer (e.g., an iPad® tablet made and sold by Apple, Inc., of Cupertino, Calif.), a dedicated display, or the like. Display 22 includes appropriate software, firmware, apps, or the like (collectively, “programming”) to display, save, manipulate, enhance, transmit, or otherwise process, images from camera 30.

System 10 may also include camera 30. As shown, camera 30 is a positionable camera that enables the clinician to position the camera 30 near the area of interest for the treatment. As shown in FIG. 5, camera 30 may comprise a flexible, positionable neck 302. For example, neck 30 may comprise jointed, flexible tubing (e.g., gooseneck tubing), or the. Camera 30 may include one or more medial cameras 304 and one or more distal cameras 306. In some embodiments, medial camera 304 and distal camera 306 may be rotatable with respect to one another, for example, via rotatable joint 308. In some embodiments, camera 30 may comprise a camera similar to those useable in endoscopy, behind-wall inspection, or the like. For example, camera 30 may comprise a camera of the types disclosed in U.S. Pat. No. 8,926,502, titled “Multi-Camera Endoscope Having A Side Service Channel,” which is hereby incorporated by reference in its entirety, or of the type known as the Triplett CobraCam 2 8115 portable inspection camera, made and sold by the Fotronic Corp., of Melrose, Mass. Other types of positionable cameras are also possible. Embodiments of camera 30 may also include a light source (not shown) that is integral with the camera 30 and may illuminate the area of interest during a procedure.

As should be understood, camera 30 communicates with (i.e., transmits to) display 22 and shows the area of interest captured by camera 30 on display 22. As discussed above, display 22 (and camera 30) may include appropriate programming to enhance, magnify, rotate, capture, record, or otherwise process the images captured by camera 30 and displayed on display 22. As should also be apparent, camera 30 may capture individual images, may show a “real time” display, or may record video images, or combinations of the foregoing. In some embodiments, communication between the camera 30 and the display 22 may be a wireless transmission.

Camera 30 and display 22 may also include programming that communicates with clinician-specific programming. For example, in a dental embodiment, camera 30 and display 22 may have programming that communicates with dental practice management tools such as Dentrix, Eaglesoft, or the like. In addition, other programming may enable communication with patients, other clinicians, specialists, or the like.

As shown in FIG. 1, system 10 may also include one or more flexible tool mounts 32. Flexible tool mounts 32 may be made of any suitable flexible mount (e.g., gooseneck tubing, or the like) and enable the clinician to holster and/or position one or more tools for use during the treatment. For example, in dental embodiments, one tool mount 32 may hold a suction instrument 34 and another may hold an ultrasonic instrument 36. Of course, other tools, for other treatments, are also possible, and more or less of the flexible mounts 32 may be included as desired.

As also shown in FIG. 1, system 10 may include a power source 38. As shown, some embodiment system 10 may include a power source 38 that is a power cord suitable for plugging into a standard wall receptacle. As shown, power source 38 may also emanate from case 12 and provide power to camera 30, display 22 and any other components that may require electric power. In other embodiments, power source 38 may comprise a battery, or the like. The battery may also be rechargeable. For embodiments of the system 10 that are built-in to chair 14, power source 38 may be provided from the same power source that powers the chair 14. Other embodiments are also possible.

FIG. 2 is a schematic back view of a portion of the system 10 in accordance with disclosed embodiments. As shown for this embodiment, case 12 may be mounted in an open position to the back of chair 14. Mounting may be accomplished in any suitable manner. For example, knob 40 may have threads that mate with a corresponding threaded portion of mounting plate 16A (shown in phantom to indicate its position behind case 12), and the case 12 may be held in position by tightening the knob 40. Other connection methods may also be used. Mounting plate 16B may be used to support articulating arm 20 (not shown on FIG. 2) as discussed above.

When in the open position shown, case 12 exposes a number of compartments 12A-D. While four compartments are shown, case 12 may have more, or less, compartments as is desired. The compartments 12A-D house components of the system 10 in an organized fashion to facilitate packing, unpacking, transporting, and the like. For example, compartment 12A may house flexible tool mounts 32, compartment 12B may house display 22 (when not mounted on articulating arm 20), compartment 12C may house the camera 30 (when not extended and in use), power source 38, and cable 26 for display 22, and compartment 12D may house articulating arm 20 (when not in use) and serve as cover for the case 12. Of course, other arrangements of components and compartments are also possible.

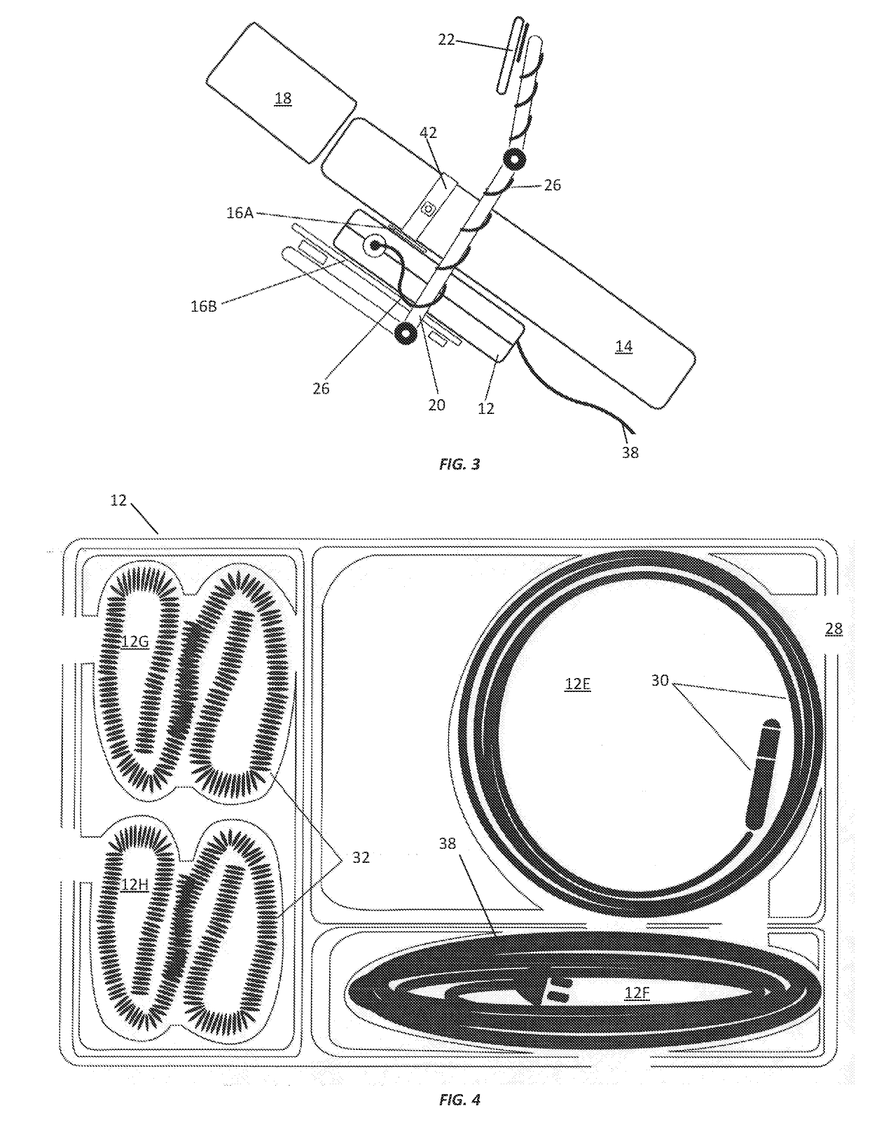

FIG. 3 is a schematic side view of the system 10 in accordance with disclosed embodiments. In this embodiment, case 12 is mounted in a closed position to mounting plate 16A which in turn is mounted to chair 14 via a clamp 42. As discussed above, clamp 42 may be any suitable clamp for selectively attaching the mounting plate 16 to the chair. For example, clamp 42 may be a C-clamp with a mouth wide enough to attach to and edge of chair 14, or clamp 42 may be a strap that fits around the entire chair 14 back, or variants of the foregoing. Attachment of system 10 to chair 14 using clamps 42 increases the portability, versatility, and ease of use of the system 10 by, among other things, increasing the types of chairs 14 the system 10 can be installed upon.

FIG. 4 is a schematic top-down, inside view of a case for the system 10 in accordance with disclosed embodiments. As shown for this embodiment, the inside of case 12 may have a number of compartments 12E-12H that are shaped to conveniently house components of the system 10. Compartments 12E-12H may be formed in any suitable manner, such as molded plastic, foam inserts, cardboard dividers, or the like. For example, compartment 12E may be shaped to house a coiled camera 30 (and optionally cable 26, not shown), compartment 12F may house power source 38, and compartments 12G-12H may be shaped to house flexible tool mounts 32. Other system components may be housed in the other side of case 12 (not shown). Other configurations of case 12 are also possible.

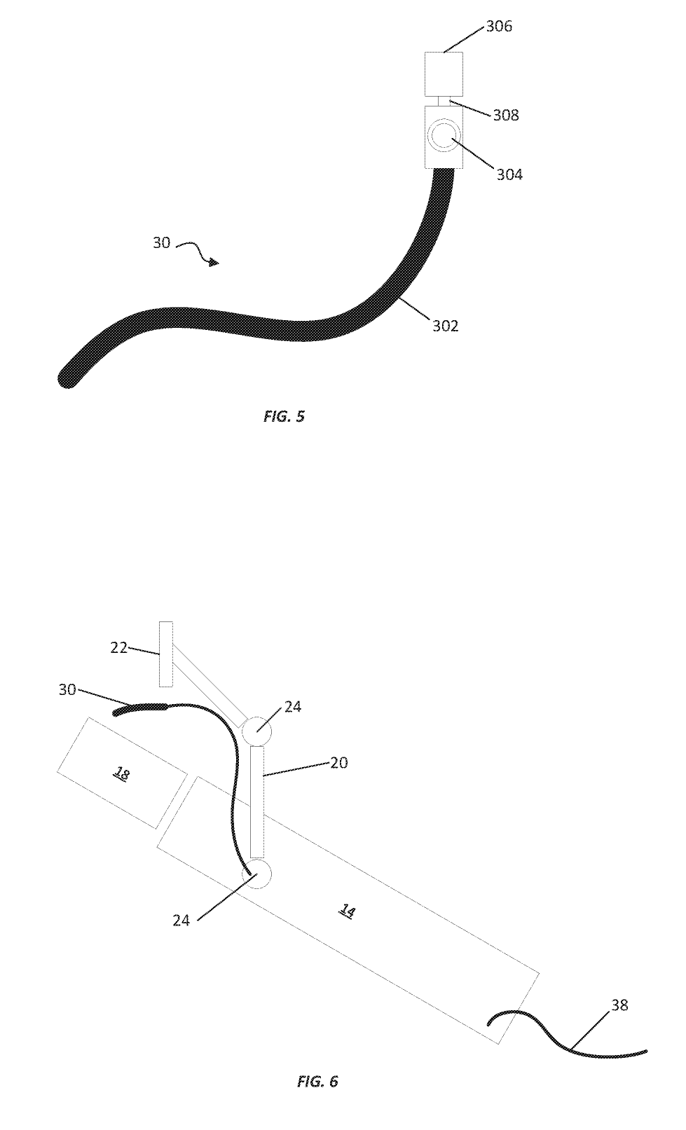

FIG. 6 is a schematic representation of the system 10 integrated into a chair 14 in accordance with disclosed embodiments. As shown, and discussed above, embodiments of system 10 may be built-into a chair 14. In such embodiments, an articulating arm 20 may extend from a side of chair 14 and provide clinician-positionable support for display 22. Likewise, flexible, positionable, camera 30 may extend from a side of chair 14 to enable the clinician to capture images of the region of interest for the treatment.

It is also possible to incorporate system 10 into the clinician's examination room. For example, the camera 30 and display 22 may be mounted at other locations than on chair 14, such as descending from the ceiling, attached to a wall, on a floor stand, or on a movable cart. Other configurations are also possible.

In use, a clinician may sit or stand next to chair 14 while a patient is seated therein, possibly in a reclined position, to receive a treatment. For embodiments with a portable system 10, prior to treatment the case 12 will have been mounted to chair 14 and power source 38 energized (e.g., plugged in). Display 22 is mounted to articulating arm 20 and camera 30 is extended to capture images of the region of interest for the treatment. Flexible tool mounts 32 may also be extended an appropriate tools (e.g., 34, 36) holstered for access during treatment. The clinician can position articulating arm 20 and display 22 in such a manner that an ergonomically sound position can be maintained by the clinician during the treatment of the patient. For example, an ergonomically sound position is one that does not require the clinician to twist and/or bend over the patient for prolonged periods of time. By positioning camera 30 in a manner that captures the region of interest, the clinician can perform the treatment while looking at display 22 and maintaining an ergonomically sound position.

Although various embodiments have been shown and described, the present disclosure is not so limited and will be understood to include all such modifications and variations are would be apparent to one skilled in the art.

What is claimed is:

1. A system comprising:
a case that is mountable to a chair;
an articulating arm that, when not in use, fits within the case and, when in use, may be attached to the chair and supports a display;
a positionable camera that, when not in use, fits within the case and, when in use, may be positioned adjacent the chair to capture images of a region of interest; and
wherein the positionable camera transmits images of the region of interest to the display.

2. The system of claim 1 further comprising:
a mounting plate that attaches to the case and enables the case to be mounted on the chair.

3. The system of claim 2 further comprising:
a clamp that attaches the mounting plate to the chair.

4. The system of claim 1 further comprising:
a flexible tool mount that, when not in use, fits within the case and, when in use, is positionable adjacent the chair and holsters a tool.

5. The system of claim 1 wherein the display comprises a general purpose tablet computer.

6. The system of claim 1 wherein the display further comprises programming for image processing.

7. The system of claim 1 wherein the positionable camera comprises an endoscopic camera.

8. The system of claim 7 wherein the endoscopic camera further comprises:
a distal camera; and
a medial camera.

9. The system of claim 8 wherein the distal camera and the medial camera are rotatable about a rotatable joint.

10. A system for ergonomic performance of dental procedures comprising:
a case that is mountable to a dentist chair;
an articulating arm that, when not in use, fits within the case and, when in use, may be attached to the dentist chair and supports a display in an ergonomically sound location;
a positionable camera that, when not in use, fits within the case and, when in use, may be positioned adjacent the dentist chair to capture images of a patient's mouth; and
wherein the positionable camera transmits images of the patient's mouth to the display.

11. The system of claim 10 further comprising:
a mounting plate that attaches to the case and enables the case to be mounted on the dentist chair.

12. The system of claim 11 further comprising:
a clamp that attaches the mounting plate to the dentist chair.

13. The system of claim 10 further comprising:
a flexible tool mount that, when not in use, fits within the case and, when in use, is positionable adjacent the dentist chair and holsters a dental tool.

14. The system of claim 10 wherein the display comprises a general purpose tablet computer.

15. The system of claim 10 wherein the display further comprises programming for image processing.

16. The system of claim 10 wherein the positionable camera comprises an endoscopic camera.

17. The system of claim 16 wherein the endoscopic camera further comprises:
a distal camera; and
a medial camera.

18. The system of claim 17 wherein the distal camera and the medial camera are rotatable about a rotatable joint.

19. A system for ergonomic performance of procedures comprising:
a chair;
an articulating arm that is attached to the chair and supports a display in an ergonomically sound location;
a positionable camera that captures images of a patient's mouth; and
wherein the positionable camera transmits images of the patient's mouth to the display.
